(19) United States
(12) Patent Application Publication
(10) Pub. No.: US 2024/0156945 A1
(43) Pub. Date: May 16, 2024

(54) HUMAN IMMUNODEFICIENCY VIRUS (HIV) ANTIGENS AND EPITOPES AND PROTEINS THAT BIND THERETO

(71) Applicants: Joseph COTROPIA, Heath, TX (US); Gaurav CHANDRA, Heath, TX (US)

(72) Inventors: Joseph COTROPIA, Heath, TX (US); Gaurav CHANDRA, Heath, TX (US)

(21) Appl. No.: 18/282,423

(22) PCT Filed: Mar. 17, 2022
(86) PCT No.: PCT/US2022/020682

§ 371 Date: Sep. 15, 2023

Related U.S. Application Data
(60) Provisional application No. 63/162,853, filed on Mar. 18, 2021.

(51) Int. Cl. A61K 39/21; A61K 39/00; C07K 14/005; C07K 16/10; C12Q 1/70; G01N 33/569

(52) U.S. Cl. CPC A61K 39/21; C07K 14/005; C07K 16/1063; C12Q 1/703; G01N 33/56988; A61K 2039/505; C12N 2740/16034; G01N 2333/162

(57) ABSTRACT

Provided are highly conserved antigens and epitopes of HIV that can be used in vaccines and to produce bindings proteins (e.g., antibodies) for detecting, treating, preventing, or reducing the risk of HIV infection and the development of AIDS.

### CROSS REFERENCE TO RELATED APPLICATIONS

This application claims priority under 35 U.S.C. § 119(e) to U.S. Provisional Application 63/162,853, filed Mar. 18, 2021, the entire contents of which are incorporated herein by reference in their entirety.

### FIELD OF INVENTION

The present disclosure relates to the field of viral vaccines, diagnostics, and therapeutics, and in particular, discloses antigens and epitopes of human immunodeficiency virus (HIV), the causative agent of acquired immunodeficiency syndrome (AIDS), that can be used in vaccines and to produce binding agents (e.g., antibodies) for treating, preventing, or reducing the risk of HIV infection and the development of AIDS, which antigens and binding proteins also can be used in assays and kits for detecting infection with HIV.

### BACKGROUND

The following discussion is merely provided to aid the reader in understanding the disclosure and is not admitted to describe or constitute prior art thereto.

The causative pathogen for Acquired Immunodeficiency Syndrome (AIDS) has been shown to be the human immunodeficiency virus (HIV). The virus gains entry into certain human lymphocytes, such as T-cells and macrophages, via the CD4 receptor. Cells which have the CD4 receptor are called CD4+ cells. T-cells and macrophages are cells which play a role in cell-mediated immunity, and the surfaces of these cells have cell surface molecules, including CD4. CD4 acts as a co-receptor to a T-cell receptor (TCR) which is involved in activating the T-cells function in immunity following an antigenic introduction to the cell.

For HIV to infect a human CD4+ cell, the virus must bind to first and second co-receptors to gain entry into the CD4+ cell and complete the cycle of infection. The gp120 protein of HIV is able to bind to CD4 (first co-receptor), after which the gp120 protein changes conformation. After this conformational change, CXCR4 and CCR5, other surface receptors on T-cells and macrophages (i.e., second co-receptors), can be bound by regions associated with gp120 and/or other HIV envelope ligands that are exposed upon the conformational change of pg120, such as gp41. Thus, binding of HIV to CD4 is the first step in a mechanism by which HIV infects T-cells, which in turn can lead to a compromised immune system that can manifest in sickness or death.

Neutralization of HIV prior to entry into CD4+ cells is a proposed approach to treating and preventing HIV infection. Neutralization of the virus can be achieved via at least two routes: an inhibitor binding directly to the virus that prevents the virus from binding to target CD4+ cells or an inhibitor binding to CD4+ cells, thereby preventing the virus from gaining access to the cells. Monoclonal antibodies designed to prevent the virus from binding with CD4+ cells have been developed. For example, antibodies previously patented by BioClonetics Incorporated can be used to disrupt fusion between HIV and the CD4+ cell membrane. See, e.g., U.S. Pat. Nos. 5,459,060, 5,777,074, 6,008,044, and 6,083,504. However, to date there are no clinically approved neutralizing antibodies or antibody-based treatments for HIV or AIDS. (A single peptide therapeutic, enfuvirtide (marketed as FUZEON® by Roche), has been approved for preventing fusion of HIV to CD4 cells.) Current standard of care therapy regimens predominately involve administration of anti-retroviral treatments such as protease inhibitors, non-nucleoside reverse transcriptase inhibitors, integrase inhibitors, and/or nucleoside/nucleotide analogues. Unlike neutralization approaches that prevent the virus from ever entering the cells, these treatments predominately rely on a mechanism of preventing viral proliferation.

Thus, there remains a need for effective agents, compositions, and methods for treating, preventing, and/or reducing the risks of HIV infection that prevent HIV from binding target cells, effectively neutralizing the virus.

### SUMMARY

Described herein are highly conserved antigens and epitopes of human immunodeficiency virus (HIV), binding agents (e.g., antibodies) that bind to the disclosed antigens and epitopes, vaccines based on the antigens, methods of treating, preventing, or reducing the risks of HIV infection with the antigens or binding proteins, and methods and kits for detecting or diagnosing infection by HIV using the antigens or binding proteins.

In one aspect, the present disclosure provides isolated peptide antigens comprising or consisting of an amino acid sequence of any one of SEQ ID NOs: 1-7. In some embodiments, the isolated peptide antigen may be recombinant.

In another aspect, the present disclosure provides vaccine compositions comprising an isolated peptide antigen comprising or consisting of an amino acid sequence of any one of SEQ ID NOs: 1-7 and a pharmaceutically acceptable carrier. In some embodiments, the isolated peptide antigen may be recombinant.

In another aspect, the present disclosure provides isolated binding proteins that bind to a conserved epitope of a HIV gp41 protein or gp120 protein, wherein the conserved epitope comprises the amino acid sequence of any one of SEQ ID NOs: 1-7. In some embodiments, the isolated binding protein prevents binding of HIV to a host cell. In some embodiments, the isolated binding protein is an antibody or an antibody fragment, including a monoclonal antibody or fragment thereof.

In another aspect, the present disclosure provides methods of reducing the risk of a HIV infection in a subject, comprising administering to the subject an effective amount of an isolated peptide antigen or a vaccine disclosed herein. In another aspect, the present disclosure provides methods of treating, preventing, or reducing the risk of a HIV infection in a subject, comprising administering to the subject a therapeutically effective amount of a binding protein disclosed herein. In some embodiments, the isolated peptide antigen or binding protein is administered by subcutaneous or intramuscular injection.

In another aspect, the present disclosure provides an in vitro method of analyzing a biological sample obtained from a subject, comprising contacting the sample with a binding protein that specifically binds to a HIV peptide antigen selected from SEQ ID NOs: 1-7, and detecting binding between the binding protein and any HIV antigen present in the sample. In some embodiments, the method comprises contacting the sample with a panel of from 2 to 7 binding proteins that each specifically binds to a different HIV peptide antigen selected from SEQ ID NOs: 1-7, and detecting binding between the binding proteins and any HIV antigen present in the sample. In some embodiments, the method may further comprise contacting the sample with a binding protein that specifically binds to SEQ ID NO: 8 and detecting binding between the binding protein and an HIV antigen comprising SEQ ID NO: 8. In some embodiments, the sample is selected from saliva, nasal fluid, nasal cells, throat cells, blood, plasma, serum, urine, and feces. In some embodiments, the subject is suspected of having a HIV infection, has been exposed to HIV, or is suspected of having been exposed to HIV. Some embodiments further comprise determining that the subject is infected with HIV when binding is detected. In some embodiments, the sample comprises biological samples obtained from a plurality of subjects. Some such embodiments further comprise determining a level of infection in the plurality of subjects.

4 In another aspect, there is provided an in vitro method of analyzing a biological sample obtained from a subject, comprising contacting the sample with a HIV peptide antigen comprising or consisting of an amino acid sequence selected from SEQ ID NOs: 1-7, and detecting binding between the peptide antigen and any anti-HIV antibodies present in the sample. In some embodiments, the method comprises contacting the sample with a panel of from 2 to 7 peptide antigens each comprising or consisting of a different an amino acid sequence selected from SEQ ID NOs: 1-7, respectively, and detecting binding between the peptide antigen and any anti-HIV antibodies present in the sample. In some embodiments, the method may further comprise contacting the sample with a peptide antigen comprising or consisting of SEQ ID NO: 8 and detecting binding between the peptide antigen and any anti-HIV antibodies that bind to SEQ ID NO: 8. In some embodiments, the sample is selected from saliva, nasal fluid, nasal cells, throat cells, blood, plasma, serum, urine, and feces. In some embodiments, the subject is suspected of having a HIV infection, has been exposed to HIV, or is suspected of having been exposed to HIV. Some embodiments further comprise determining that the subject is infected with HIV when binding is detected. In some embodiments, the sample comprises biological samples obtained from a plurality of subjects. Some such embodiments further comprise determining a level of infection in the plurality of subjects.

In another aspect, the present disclosure also provides in vitro methods of analyzing a biological sample obtained from a subject, comprising extracting nucleic acids from the biological sample, contacting the extracted nucleic acids with a pair of primers that specifically amplify a nucleic acid sequence encoding a peptide of any one of SEQ ID NOs: 1-7, and detecting the presence of any amplified nucleic acid sequence present in the sample. The methods may comprise contacting the sample with a panel of from 2 to 7 primer pairs each specific for a nucleic acid sequence that encodes a different amino acid sequence selected from SEQ ID NOs: 1-7, and detecting amplification of each nucleic acid sequence if present in the sample. In some embodiments, the method may further comprise contacting the sample with a primer pair specific for a nucleic acid sequence that encodes SEQ ID NO: 8 and detecting amplification of the nucleic acid sequence if present in the sample. In some embodiments, the sample may be selected from saliva, nasal fluid, nasal cells, throat cells, blood, plasma, serum, urine, and feces. In some embodiments, the subject is suspected of having a HIV infection, has been exposed to HIV, or is suspected of having been exposed to HIV. Some embodiments further comprise determining that the subject is infected with HIV when amplification is detected. In some embodiments, the sample comprises biological samples obtained from a plurality of subjects. Some such embodiments further comprise determining a level of infection in the plurality of subjects.

In another aspect, the present disclosure provides kits comprising one or more binding proteins that each specifically binds to a different peptide comprising or consisting of any one of SEQ ID NOs: 1-7, a solid substrate to which the one or more binding proteins is attached, and a detectably labeled antibody that specifically binds to the peptide to which the one or more binding proteins specifically binds. In some embodiments, the kit may further comprise a binding protein that specifically binds to a peptide comprising or consisting of SEQ ID NO: 8. In another aspect, the present disclosure provides kits comprising one or more peptides each comprising or consisting of a different one of SEQ ID NOs: 1-7, a solid substrate to which the one or more peptides is attached, and a detectably labeled antibody that specifically binds to IgE or IgD, wherein the IgE or IgD are optionally human. In some embodiments, the kit may further comprise a peptide comprising or consisting of SEQ ID NO: 8. In some embodiments, the solid substrate is selected from a bead, a plate, a well, a dish, a slide, or a strip.

In another aspect, the present disclosure provides kits comprising one or more primer pairs each capable of specifically amplifying a nucleic acid sequence that encodes a different peptide selected from SEQ ID NOs: 1-7, wherein: (a) at least one primer of the primer pair is detectably labeled; or (b) the kit further comprises a detectably labeled probe that hybridizes to the nucleic acid sequence amplified by the primer pair. In some embodiments, the kit comprises 1, 2, 3, 4, 5, 6, 7, or 8 primer pairs, each primer pair being capable of specifically amplifying a nucleic acid sequence that encodes a different peptide selected from SEQ ID NOs: 1-7, and, optionally a primer pair capable of amplifying a nucleic acid sequence encoding a peptide comprising or consisting of SEQ ID NO: 8.

In another aspect, the present disclosure provides isolated peptide antigens or vaccines disclosed herein for inducing an immune response to HIV. In some embodiments, the isolated peptide antigen may be recombinant.

In another aspect, the present disclosure provides isolated binding proteins disclosed herein for treating, preventing, or reducing the risk of a HIV infection in a subject in need thereof. In some embodiments, the isolated binding protein prevents the HIV from binding to a host cell. In some embodiments, the binding protein is an antibody or an antibody fragment, including a monoclonal antibody or fragment thereof.

In another aspect, the present disclosure provides uses of isolated peptide antigens disclosed herein in the preparation of a vaccine for inducing an immune response to HIV. In some embodiments, the peptide antigen may be recombinant.

In another aspect, the present disclosure provides uses of isolated binding proteins disclosed herein in the preparation of a medicament for treating, preventing, or reducing the risk of a HIV infection in a subject in need thereof. In some embodiments, the binding protein prevents the HIV from binding to a host cell. In some embodiments, the binding protein is an antibody or an antibody fragment.

In another aspect, the present disclosure provides methods of preparing an antibody that binds to a peptide antigen or epitope comprising or consisting of an amino acid sequence of any one of SEQ ID NOs: 1-7, comprising: (a) identifying an asymptomatic patient that has been infected with HIV as a donor for obtaining immune B-lymphocytes that produce high titers of HIV-neutralizing antibodies; (b) collecting the B-lymphocytes from the patient; (c) immortalizing the B-lymphocytes; (d) collecting antibodies produced by the immortalized B-lymphocytes; and (e) screening the antibodies for binding to the peptide antigen of any one of SEQ ID NOs: 1-7. In some embodiments, the method may further comprise testing the antibodies for binding to HIV. In some embodiments, the method may further comprise epitope mapping the antibodies that tested positive for binding to HIV. In some embodiments, immortalizing the B-lymphocytes comprises fusing a B-lymphocyte with a heteromyeloma cell in order to produce a heterohybridoma cell.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the percentage of conservation of the disclosed gp41 and gp120 epitopes across all 87,336 viral isolates of HIV.

### DETAILED DESCRIPTION

The present disclosure provides four highly conserved antigens and epitopes of the human immunodeficiency virus (HIV) gp41 transmembrane protein (i.e., “gp41”) and three highly conserved antigens and epitopes of the HIV gp120 transmembrane protein (i.e., “gp120”). The disclosed antigens and epitopes are highly conserved across all HIV isolates (clades and groups), indicating that there is strong selective pressure to maintain these sequences, even as variants of HIV develop. As a result, agents (e.g. antibodies) that bind to the disclosed antigens and epitopes may provide pan-protection, both in terms of treatment and prevention, against variant strains of HIV, and potentially other lentiviruses or retroviruses that share the same or similar conserved sequences. Similarly, vaccines based on the disclosed highly conserved antigens may provide broadly universal and temporally durable protection against a range of HIV variants and lentivirus or retrovirus strains. Also, diagnostic methods and kits based on the disclosed antigens may permit detection of infection by known and future strains of HIV.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein. 28 As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” is intended to mean that the compositions and methods include the recited elements, but does not exclude others.

An used herein, the term “isolated” when used in the context of referring to a peptide antigen or binding protein or antibody as discussed herein refers to one which has been separated from at least some of the components with which it existed in nature (for those isolated from nature) or with which it was produced (for those produced, e.g., in a laboratory setting).

As used herein, a “variant” when used in the context of referring to a peptide means a peptide sequence that is derived from a parent sequence by incorporating one or more amino acid changes, which can include substitutions, deletions, or insertions. For the purposes of this disclosure, a variant may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) sequence. For purposes of this disclosure, the terms “variant” and “derivative” when used in the context of referring to a peptide are used interchangeably.

As used herein, the phrases “effective amount,” “therapeutically effective amount,” and “therapeutic level” mean the dosage or concentration of an antigen, antibody or binding protein that provides the specific pharmacological effect for which the antigen, antibody or binding protein is administered in a subject in need of such treatment, e.g., to induce a protective immune response against HIV or to treat or prevent a HIV infection (e.g., AIDS). It is emphasized that a therapeutically effective amount or therapeutic level of an antigen, antibody or binding protein will not always be effective in inducing a protective immune response or treating or preventing the infections described herein, even though such dosage is deemed to be a therapeutically effective amount by those of skill in the art. The therapeutically effective amount may vary based on the route of administration and dosage form, the age and weight of the subject, and/or the subject's condition, including the severity of the HIV infection.

The terms “treat,” “treatment” or “treating” as used herein with reference to a HIV infection refer to reducing or eliminating viral load, including reducing viral load to an undetectable level.

The terms “prevent,” “preventing” or “prevention” as used herein with reference to a HIV infections refer to precluding or reducing the risk of an infection from developing in a subject exposed to a HIV, or to precluding or reducing the risk of developing a high viral load of HIV. Prevention may also refer to the prevention of a subsequent infection once an initial infection has been treated or cured.

The terms “individual,” “subject,” and “patient” are used interchangeably herein, and refer to any individual mammalian subject, e.g., bovine, canine, feline, equine, or human. In specific embodiments, the subject, individual, or patient is a human.

### II. HIV, gp41, and gp120

HIV is a virus spread through certain body fluids that attacks the body's immune system, specifically the CD4 cells, often called T cells. HIV reduces the number of CD4 cells (helper T4-cells) in the body. Over time, HIV can destroy so many of these cells that the body cannot fight off other infections and diseases. Opportunistic infections or cancers take advantage of the very weak immune system and signal that the person has AIDS.

When an individual is infected with HIV and does not receive treatment, the individual will typically progress through three stages of disease. Conventional HIV therapy, known as anti-retroviral therapy, can be beneficial at all stages of the disease if taken the right way, every day. Anti-retroviral treatment can slow or prevent progression from one stage to the next, and can reduce the chance of transmitting HIV to someone else. However, there is still no currently available cure or prophylactic for HIV infection.

Stage 1: Acute HIV infection: Within 2 to 4 weeks after infection with HIV, individuals may experience a flu-like illness, which may last for a few weeks. This is the body's natural response to infection. When individuals have acute HIV infection, they have a large amount of virus in their blood and are very contagious. But people with acute infection are often unaware that they are infected because they may not feel sick right away or at all. Treating HIV at this stage, or preventing infection altogether, would provide dramatic clinical and societal benefit.

Stage 2: Clinical latency (HIV inactivity or dormancy): This period is sometimes called asymptomatic HIV infection or chronic HIV infection. During this phase, HIV is still active but reproduces at very low levels. People may not have any symptoms or get sick during this time. For people who are not taking medicine to treat HIV, this period can last a decade or longer, but some may progress through this phase faster. People who are taking medicine to treat HIV (e.g., anti-retroviral therapy) may be in this stage for several decades.

People can still transmit HIV to others during this phase, although people who are on anti-retroviral therapy and stay virally suppressed (having a very low level of virus in their blood) are much less likely to transmit HIV than those who are not virally suppressed. At the end of this phase, a person's viral load starts to go up, and CD4 cell count begins to go down. As this happens, the person may begin to have symptoms as the virus levels increase in the body, and the person moves into Stage 3.

Stage 3: Acquired immunodeficiency syndrome (AIDS): AIDS is the most severe phase of HIV infection. People with AIDS have such badly damaged immune systems that they may contract an increasing number of severe illnesses (i.e., opportunistic illnesses) that may be debilitating or fatal. Without treatment, people with AIDS typically survive only about 3 years. Common symptoms of AIDS include chills, fever, sweats, swollen lymph glands, weakness, and weight loss. People typically are diagnosed with AIDS when their CD4 cell count drops below 200 cells/mm³ of blood or if they develop certain opportunistic illnesses. People with AIDS can have a high viral load and may be very infectious.

Mechanism of entry: The Envelope glycoprotein (Env) of HIV is arranged on the surface of the virus and virus-infected cells as a hetero-trimer. Each monomer is composed of a receptor-binding surface unit (gp120) and a fusogenic gp41 transmembrane unit. Gp41 and gp120 are encoded together as one gp160 by the env gene of HIV. Gp160 is then extensively glycosylated and proteolytically cleaved by furin, a host cellular protease, to produce gp120 (which includes amino acids 1-511 of gp160) and gp41 (which includes amino acids 512-856 of gp160). The gp120 subunit binds to CD4 and a coreceptor either CXCR4 or CCR5 on T helper cells. Binding of HIV gp120 to CD4 triggers a complex sequence of events involving several conformational changes in gp120 that result in exposure of coreceptor binding sites on gp120 and the N-terminal and C-terminal heptad repeat regions of gp41. Following engagement of gp120 with coreceptor, the gp41 heptad repeat domains interact with each other to form a six helix bundle catalyzing fusion of target and viral membranes. In other words, HIV gp41 is integral to HIV binding and entry into human cells. Accordingly, targeting gp41 with binding proteins (e.g., antibodies) or vaccines that would produce antibodies in vivo would neutralize the virus and prevent its entry in human CD4 cells.

The present disclosure provides highly conserved antigens and epitopes of HIV gp41 and gp120 that can be used in vaccines or in the preparation of binding proteins for treating and/or preventing HIV infection.

### III. Antigens and Epitopes of gp41

The present disclosure provides highly conserved peptide sequences of the HIV gp41 protein and the gp120 protein that can be used as antigens or epitopes of binding agents (e.g., binding proteins such as antibodies) for vaccines, targets for drugs, and/or for treating and/or preventing HIV infection.

The disclosed epitopes were discovered using computational prediction and machine learning approaches to assess and compare 87,336 viral isolates to identify highly conserved regions. The pronounced conservation of these regions across this many isolates indicates that these regions are unlikely to mutate as HIV evolves and spawns new variants. The conserved nature of these epitopes also indicates that antibodies or other binding agents that bind to these regions and vaccines that contain an antigen that comprises or consists of a peptide having one of the amino acid sequences may provide broad therapeutic treatment and/or protection against a wide range of lentiviruses, particularly HIV, and that these epitopes and antibodies or other binding proteins that bind to these regions can be used in diagnostic assays useful for detecting or diagnosing HIV infection on an individual or community level.

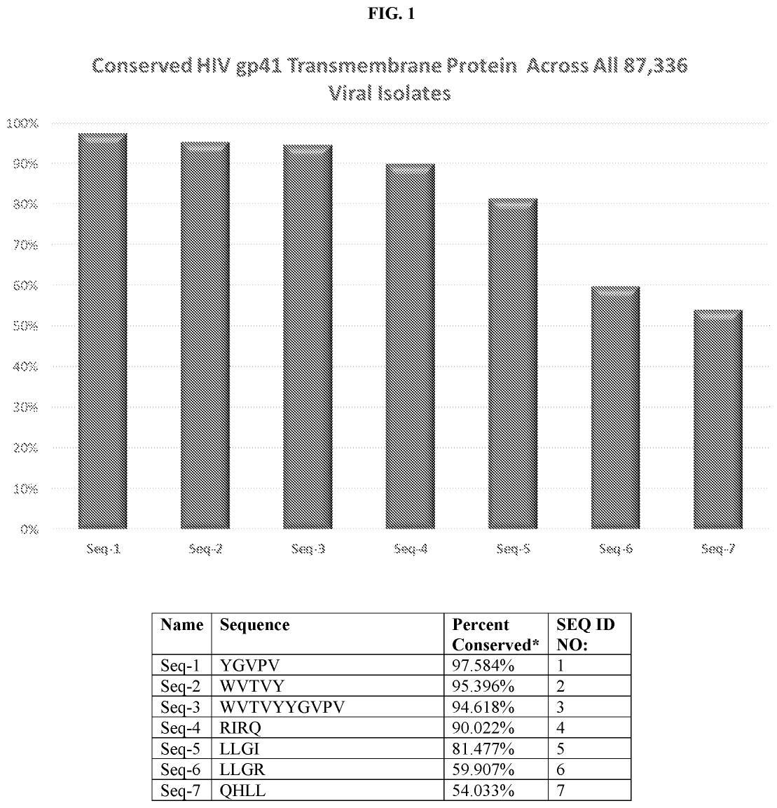

Specific antigens/epitopes of gp41 of HIV disclosed herein are shown in Table 1 below; the percent conservation across isolates also is shown in FIG. 1.

TABLE 1
HIV gp41 Antigens/Epitopes

| | | Residues | Percent | SEQ |
|---|---|---|---|---|
| Name | Sequence | in gp160 | Conserved* | ID NO: |
| Seq-1 | YGVPV | 40-44 (gp120) | 97.584% | 1 |
| Seq-2 | WVTVY | 35-39 (gp120) | 95.396% | 2 |
| Seq-3 | WVTVY | 35-44 (gp120) | 94.618% | 3 |
| | YGVPV | | | |
| Seq-4 | RIRQ | 846-849 (gp41) | 90.022% | 4 |
| Seq-5 | LLGI | 592-595 (gp41) | 81.477% | 5 |
| Seq-6 | LLGR | 784-787 (gp41) | 59.907% | 6 |
| Seq-7 | QHLL | 563-566 (gp41) | 54.033% | 7 |

*Percent conserved across 87,336 viral isolates.

Three-dimensional artificial intelligence analysis of the HIV gp120/gp41 protein was undertaken to identify accessible regions for antibody binding, and it was determined that all of the disclosed epitopes are linear and conserved epitopes that are accessible by antibodies and unaffected by mutations. In other words, the disclosed sequences appear to be immutable, which makes these sequences particularly useful for prophylactic, therapeutic, and diagnostic purposes, as HIV is characterized as having a high rate of mutations and individual isolates of the virus will often carry multiple mutations. Thus, sequences that remain the same across isolates may provide broad spectrum and sustained utility that has been previously difficult to achieve.

The peptide sequences disclosed in Table 1 can be used as antigens in a vaccine or can be used to develop a binding agent, such as a binding protein, such as an antibody, to be used in passive immunization or therapy. Any of the peptide sequences in Table 1 (i.e., SEQ ID NOs: 1-7) can be formulated in a vaccine to immunize a subject and/or to induce an immune response (e.g., to induce antibodies) in the subject, and can be used singly or in combination (e.g., in multivalent vaccines or multivalent diagnostics as discussed below). Alternatively, the peptide sequences in Table 1 (i.e., SEQ ID NOs: 1-7) can be used to produce (raise) antibodies, such as by immunizing an animal, such as a mouse or rat or human, to produce (raise) antibodies that specifically bind to the immunizing peptide sequence, including to produce monoclonal antibodies. The antibodies induced or produced by such methods will bind to the corresponding sequence on the gp41 or gp120 proteins, e.g., will bind to that epitope on the gp41 or gp120 proteins, and so may be used in therapeutic treatment and prophylactic protocols. Additionally, monoclonal antibodies specific to these HIV antigens may be produced in humans-especially in long-term non-progressors—and therefore can also be derived extracorporally from human immune B-cells that have been elicited as a result of natural HIV infection. See, e.g., U.S. Pat. No. 5,777,074. Such antibodies can be used singly or in combination for analytical purposes or in passive immunotherapy as discussed below.

### IV. HIV Vaccines

The present disclosure provides vaccines comprising one or more antigen(s) that comprise or consist of a peptide having one of the amino acid sequences in Table 1 (i.e., SEQ ID NOs: 1-7). The antigens can be prepared by methods known in the art, such as chemical synthesis, or by recombinant methods. Techniques for making peptides are known in the art, and can be used to obtain antigens as disclosed herein. The vaccines may comprise one or more antigen(s) formulated in a pharmaceutically acceptable carrier for the intended route of administration, as discussed in more detail below.

The immune response elicited by immunization with a vaccine as disclosed herein (e.g., comprising one or more antigen(s) that comprise or consist of a peptide having one of the sequences in Table 1 (i.e., SEQ ID NOs: 1-7)) is expected to induce production of antibodies that bind highly conserved epitopes of HIV and provide broad spectrum immune protection against HIV and variants thereof.

A vaccine as disclosed herein, comprising one or more antigen(s) that comprise or consist of one or more of the peptide sequences in Table 1 (i.e., comprising or consisting of at least one of SEQ ID NOs: 1-7) can be used for treating or preventing a HIV infection (e.g., AIDS) or other similar lentiviruses and/or retroviruses. Optimal doses and routes of administration may vary, such as based on the route of administration and dosage form, the age and weight of the subject, and/or the subject's condition, and can be determined by the skilled practitioner. The vaccine may be formulated for injection and administered parenterally, such as intramuscularly, subcutaneously, or intradermally. The vaccine may be formulated for intravenous injection or infusion. The disclosed vaccines may be formulated to be administered alone or concurrently with another therapeutic agent for treating HIV. The vaccines may be formulated to be administered in sequence with another therapeutic agent. For example, the vaccine may be administered either before or after the subject has received a regimen of an anti-viral therapy. The vaccines may be administered as a single dose or an initial dose followed by one or more booster doses.

A vaccine as disclosed herein, comprising one or more antigen(s) that comprise or consist of one or more of the peptide sequences in Table 1 (i.e., comprising or consisting of at least one of SEQ ID NOs: 1-7), and, optionally SEQ ID NO:8, can be formulated or administered with an adjuvant to improve immune responses and promote protective responses. An adjuvant is an ingredient used in some vaccines that helps create a stronger immune response in people receiving the vaccine. Adjuvants help the body to produce an immune response strong enough to protect the person from the disease he or she is being vaccinated against. Those skilled in the art are aware of pharmaceutically acceptable adjuvants that may be combined with one or more of the disclosed antigens to prepare a vaccine.

### V. HIV Binding Proteins

The present disclosure also provides binding proteins, such as antibodies, including monoclonal antibodies, that specifically bind to the disclosed antigen/epitope sequences (SEQ ID NOs: 1-7). While not wanting to be bound by theory, the disclosed binding proteins can prevent the gp41 protein from fusing to the membrane of HIV and host cells. The disclosed binding proteins can be used for passive immunization or as therapeutics. The disclosed binding proteins can be used in methods for diagnosing, treating, preventing, or reducing the risk of HIV infection (e.g., AIDS) or the development of a HIV infection in an individual in need thereof. For example, the disclosed binding proteins can be administered in a therapeutically effective amount to a subject in need thereof to reduce circulating levels of HIV, reduce viral load, and/or reduce, ameliorate, or eliminate one or more signs or symptoms of HIV infection.

The disclosed binding proteins include antibodies and antibody fragments, monomers, dimers, single-domain antibodies, and other immunoglobulin fragments, variants, or derivatives. The binding proteins disclosed herein can be obtained by any means, including from in vitro sources (e.g., a hybridoma or a cell line producing the peptide recombinantly) and in vivo sources (e.g., rodents, rabbits, humans, etc.). In some embodiments, the binding proteins may be produced by a heterohybridoma, as discussed in more detail below. See also U.S. Pat. No. 5,777,074. In some embodiments, the binding protein may be a monoclonal antibody.

The binding proteins disclosed herein specifically bind to an epitope on the HIV gp41 protein disclosed herein (SEQ ID NOs: 1-7). In some embodiments, SEQ ID NOs: 1-7 may represent the minimal epitope to which the disclosed binding proteins specifically bind, i.e., the minimal essential core epitope(s).

In general, the disclosed binding proteins comprise at least a least a portion of an immunoglobulin heavy chain. For instance, in some embodiments, the binding protein may comprise a heavy chain monomer, a heavy chain dimer, or may be a single-domain antibody (i.e., a V_{H}H fragment, a “nanobody,” or a “camelid-like” antibody). A single-domain antibody may comprise or consist of a V_{H} domain, a C_{H2} domain, and a C_{H3} domain, but not a V_{K} domain or a C_{H1} domain.

The disclosed binding proteins can comprise, but do not require, an immunoglobulin light chain in order to bind a HIV gp41 protein epitope disclosed herein. In some embodiments, the disclosed binding proteins comprise both a heavy and light chain. In some embodiments, the disclosed binding proteins are full antibodies (e.g., complete IgGs). Human, partially humanized, fully humanized, and chimeric versions of the binding protein disclosed can be made by methods known in the art, such as using a transgenic animal (e.g., a mouse) wherein one or more endogenous immunoglobulin gene sequences are replaced with one or more human immunoglobulin gene sequences. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9): 1117-25 (2005), and Lonberg, Handb. Exp. Pharmacol., 181: 69-97 (2008)).

The disclosed binding proteins may be an antibody. Typically, an antibody consists of four polypeptides: two identical copies of a heavy (H) chain polypeptide and two copies of a light (L) chain polypeptide. Each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody, however, some of the disclosed peptides may comprise a heavy chain without a light chain. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The disclosed binding proteins may be an “antibody fragment,” which refers to one or more portions of a HIV-binding antibody that exhibits the ability to bind to an epitope on the HIV gp41 protein defined by any one of SEQ ID NOs: 1-7. Examples of binding fragments include (i) Fab fragments (monovalent fragments consisting of the V_{L}, V_{H}, C_{L} and C_{H1} domains); (ii) F(ab′)2 fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDRs), e.g., V_{H} CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples of types of antibody fragments include HIV-binding domain immunoglobulin fusion proteins comprising (i) a HIV-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization.

As noted above, the disclosed binding proteins may or may not comprise a light chain. Similarly, he disclosed binding proteins may or may not comprise a CH1 region. For instance, in some embodiments, a binding protein may comprise or consist of a V_{H} domain, a C_{H2} domain, and a C_{H3} domain. In some embodiments, a binding protein may comprise or consist of a V_{H} domain, a C_{H1} domain, a C_{H2} domain, and a C_{H3} domain. In some embodiments, the constant domains may comprise one or more modifications, such as an amino acid substitution.

The disclosed binding proteins include monoclonal antibodies (mAbs) and fragments thereof, which may be obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with a peptide antigen, such as a peptide antigen comprising any one of SEQ ID NOs: 1-7; or created from “immune B-cells” obtained from convalescent HIV patients' peripheral mononuclear cells.

Recombinant versions of the disclosed binding proteins may be obtained by methods known in the art, for example, using phage display technologies, yeast surface display technologies (Chao et al., Nat. Protoc., 1(2): 755-68 (2006)), mammalian cell surface display technologies (Beerli et al., PNAS, 105(38): 14336-41 (2008), and/or by expressing or co-expressing component polypeptides, such as heavy and light chain polypeptides. Other techniques for making peptides and antibodies are known in the art, and can be used to obtain binding proteins as well.

The disclosed binding proteins may be or be derived from a human IgG1 antibody, a human IgG2 antibody, a human IgG3 antibody, or a human IgG4 antibody. In some embodiments, the binding protein may be or be derived from a class of antibody selected from IgG, IgM, IgA, IgE, and IgD. That is, the disclosed binding proteins may comprise all or part of the constant regions, framework regions, or a combination thereof of an IgG, IgM, IgA, IgE, or IgD antibody. For instance, a disclosed binding protein comprising an IgG1 immunoglobulin structure may be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). A person of ordinary skill in the art will understand that, for example, a recombinant IgG1 immunoglobulin structure can be “switched” to the corresponding regions of immunoglobulin structures from other immunoglobulin classes, such as recombinant secretory IgA1 or recombinant secretory IgA2, such as may be useful for topical application onto mucosal surfaces. For example, immunoglobulin IgA structures are known to have applications in protective immune surveillance directed against invasion of infectious diseases, which makes such structures suitable for methods of using the disclosed binding proteins in such contexts, e.g., treating or preventing HIV infection (e.g., AIDS) or the spread of HIV from one individual to another.

In some embodiments, a disclosed binding protein may comprise one or more mutations, alterations, or modifications that improve one or more properties or functions of the binding protein. Such mutations, alterations, or modifications may comprise, for example, changes to the Fc region to increase the ability of the peptide to mediate cellular cytotoxicity functions like antibody dependent cell cytotoxicity (ADCC), antibody dependent cell mediated phagocytosis (ADCP), and/or complement fixation (CDC). A wide number of mutations to the Fc domain that enhance binding to Fc receptors have been reported, for example, S239D/A330L/I332E, F243L, and G236A. Additionally or alternatively, mutations to the Fc region that increase the circulating half-life of a disclosed HIV-binding peptide may be incorporated into the structure. For example, mutations to engineer the pH-dependent interaction of the Fc domain with FcRn to increase affinity at pH 6.0 while retaining minimal binding at pH 7.4, can increase half-life and improve efficacy under physiological conditions. Exemplary mutations that may be incorporated in order to enhance C1q receptor or Fc receptor binding are shown in the table below.

TABLE 2
Potential Fc Mutations

| Mutation | Mechanism of Action | Effect |
|---|---|---|
| S267E | Enhance C1q binding | Increase CDC |
| H268F/S324T | Enhance C1q and Fc | Increase CDC, |
| | receptor binding | ADCC, ADCP |
| H268F/S324T/G236A/I332E | Enhance C1q and Fc | Increase CDC, |
| | receptor binding | ADCC, ADCP |
| H268F/S324T/G236D/I332E | Enhance C1q and Fc | Increase CDC, |
| | receptor binding | ADCC, ADCP |
| K326A/E333A | Enhance C1q and Fc | Increase CDC, |
| | receptor binding | ADCC |
| E345R | Enhance multimerization | Increase CDC, |
| | | ADCC, ADCP |
| M252Y/S254T/T256E | Enhanced Fc receptor | Increased |
| | binding at pH 6.0 | half-life |
| T250Q/M428L | Enhanced Fc receptor | Increased |
| | binding at pH 6.0 | half-life |
| P230S/N315D/M428L/N434Y | Enhanced Fc receptor | Increased |
| | binding at pH 6.0 | half-life |

In some embodiments, the disclosed binding proteins may be conjugated to polyethylene glycol (PEG) and/or albumin, which may increase the half-life and decrease the potential immunogenicity of the peptide.

The disclosed binding proteins may bind to a conserved HIV epitope as disclosed herein (e.g., any one of SEQ ID NOs: 1-7) with a high affinity. For example, the disclosed binding proteins and antibodies can have a K_{D} of at least 3.0×10⁻⁸, at least 2.5×10⁻⁸, at least 2.0×10⁻⁸, at least 1.5×10⁻⁸, at least 1.0×10⁻⁸, at least 0.5×10⁻⁸, at least 9.95×10⁻⁹, at least 9.90×10⁻⁹, at least 9.85×10⁻⁹, at least 9.80×10⁻⁹, at least 9.75×10⁻⁹, at least 9.70×10⁻⁹, at least 9.65×10⁻⁹, at least 9.60×10⁻⁹, at least 9.55×10⁻⁹, at least 9.5×10⁻⁹, at least 9.45×10⁻⁹, at least 9.40×10⁻⁹, at least 9.35×10⁻⁹, at least 9.30×10⁻⁹, at least 9.25×10⁻⁹, at least 9.20×10⁻⁹, at least 9.15×10⁻⁹, at least 9.10×10⁻⁹, at least 9.05×10⁻⁹, at least 9.0×10⁻⁹, at least 8.95×10⁻⁹, at least 8.90×10⁻⁹, at least 8.85×10⁻⁹, at least 8.80×10⁻⁹, at least 8.75×10⁻⁹, at least 8.70×10⁻⁹, at least 8.65×10⁻⁹, at least 8.60×10⁻⁹, at least 8.55×10⁻⁹, at least 8.5×10⁻⁹, at least 8.45×10⁻⁹, at least 8.40×10⁻⁹, at least 8.35×10⁻⁹, at least 8.30×10⁻⁹, at least 8.25×10⁻⁹, at least 8.20×10⁻⁹, at least 8.15×10⁻⁹, at least 8.10×10⁻⁹, at least 8.05×10⁻⁹, at least 8.0×10⁻⁹, at least 7.95×10⁻⁹, at least 7.90×10⁻⁹, at least 7.85×10⁻⁹, at least 7.80×10⁻⁹, at least 7.75×10⁻⁹, at least 7.70×10⁻⁹, at least 7.65×10⁻⁹, at least 7.60×10⁻⁹, at least 7.55×10⁻⁹, at least 7.5×10⁻⁹, at least 7.45×10⁻⁹, at least 7.40×10⁻⁹, at least 7.35×10⁻⁹, at least 7.30×10⁻⁹, at least 7.25×10⁻⁹, at least 7.20×10⁻⁹, at least 7.15×10⁻⁹, at least 7.10×10⁻⁹, at least 7.05×10⁻⁹, at least 7.0×10⁻⁹, at least 6.95×10⁻⁹, at least 6.90×10⁻⁹, at least 6.85×10⁻⁹, at least 6.80×10⁻⁹, at least 6.75×10⁻⁹, at least 6.70×10⁻⁹, at least 6.65×10⁻⁹, at least 6.60×10⁻⁹, at least 6.55×10⁻⁹, at least 6.5×10⁻⁹, at least 6.45×10⁻⁹, at least 6.40×10⁻⁹, at least 6.35×10⁻⁹, at least 6.30×10⁻⁹, at least 6.25×10⁻⁹, at least 6.20×10⁻⁹, at least 6.15×10⁻⁹, at least 6.10×10⁻⁹, at least 6.05×10⁻⁹, at least 6.0×10⁻⁹, at least 5.95×10⁻⁹, at least 5.90×10⁻⁹, at least 5.85×10⁻⁹, at least 5.80×10⁻⁹, at least 5.75×10⁻⁹, at least 5.70×10⁻⁹, at least 5.65×10⁻⁹, at least 5.60×10⁻⁹, at least 5.55×10⁻⁹, at least 5.5×10⁻⁹, at least 5.45×10⁻⁹, at least 5.40×10⁻⁹, at least 5.35×10⁻⁹, at least 5.30×10⁻⁹, at least 5.25×10⁻⁹, at least 5.20×10⁻⁹, at least 5.15×10⁻⁹, at least 5.10×10⁻⁹, at least 5.05×10⁻⁹, at least 5.0×10⁻⁹, at least 4.95×10⁻⁹, at least 4.90×10⁻⁹, at least 4.85×10⁻⁹, at least 4.80×10⁻⁹, at least 4.75×10⁻⁹, at least 4.70×10⁻⁹, at least 4.65×10⁻⁹, at least 4.60×10⁻⁹, at least 4.55×10⁻⁹, at least 4.5×10⁻⁹, at least 4.45×10⁻⁹, at least 4.40×10⁻⁹, at least 4.35×10⁻⁹, at least 4.30×10⁻⁹, at least 4.25×10⁻⁹, at least 4.20×10⁻⁹, at least 4.15×10⁻⁹, at least 4.10×10⁻⁹, at least 4.05×10⁻⁹, at least 4.0×10⁻⁹, at least 3.95×10⁻⁹, at least 3.90×10⁻⁹, at least 3.85×10⁻⁹, at least 3.80×10⁻⁹, at least 3.75×10⁻⁹, at least 3.70×10⁻⁹, at least 3.65×10⁻⁹, at least 3.60×10⁻⁹, at least 3.55×10⁻⁹, at least 3.5×10⁻⁹, at least 3.45×10⁻⁹, at least 3.40×10⁻⁹, at least 3.35×10⁻⁹, at least 3.30×10⁻⁹, at least 3.25×10⁻⁹, at least 3.20×10⁻⁹, at least 3.15×10⁻⁹, at least 3.10×10⁻⁹, at least 3.05×10⁻⁹, at least 3.0×10⁻⁹, at least 2.95×10⁻⁹, at least 2.90×10⁻⁹, at least 2.85×10⁻⁹, at least 2.80×10⁻⁹, at least 2.75×10⁻⁹, at least 2.70×10⁻⁹, at least 2.65×10⁻⁹, at least 2.60×10⁻⁹, at least 2.55×10⁻⁹, at least 2.5×10⁻⁹, at least 2.45×10⁻⁹, at least 2.40×10⁻⁹, at least 2.35×10⁻⁹, at least 2.30×10⁻⁹, at least 2.25×10⁻⁹, at least 2.20×10⁻⁹, at least 2.15×10⁻⁹, at least 2.10×10⁻⁹, at least 2.05×10⁻⁹, at least 2.0×10⁻⁹, at least 1.95×10⁻⁹, at least 1.90×10⁻⁹, at least 1.85×10⁻⁹, at least 1.80×10⁻⁹, at least 1.75×10⁻⁹, at least 1.70×10⁻⁹, at least 1.65×10⁻⁹, at least 1.60×10⁻⁹, at least 1.55×10⁻⁹, at least 1.5×10⁻⁹, at least 1.45×10⁻⁹, at least 1.40×10⁻⁹, at least 1.35×10⁻⁹, at least 1.30×10⁻⁹, at least 1.25×10⁻⁹, at least 1.20×10⁻⁹, at least 1.15×10⁻⁹, at least 1.10×10⁻⁹, at least 1.05×10⁻⁹, at least 1.0×10⁻⁹, at least 0.95×10⁻⁹, at least 0.90×10⁻⁹, at least 0.85×10⁻⁹, at least 0.80×10⁻⁹, at least 0.75×10⁻⁹, at least 0.70×10⁻⁹, at least 0.65×10⁻⁹, at least 0.60×10⁻⁹, at least 0.55×10⁻⁹, at least 0.5×10⁻⁹, at least 0.45×10⁻⁹, at least 0.40×10⁻⁹, at least 0.35×10⁻⁹, at least 0.30×10⁻⁹, at least 0.25×10⁻⁹, at least 0.20×10⁻⁹, at least 0.15×10⁻⁹, at least 0.10×10⁹, at least 0.05×10⁹, at least 9.5×10⁻¹⁰, at least 9.0×10⁻¹⁰, at least 8.5×10⁻¹⁰, at least 8.0×10⁻¹⁰, or any value in between.

Any of the binding proteins or antibodies disclosed herein can be used for treating and/or preventing a HIV infection, such as AIDS. Optimal doses and routes of administration may vary, such as based on the route of administration and dosage form, the age and weight of the subject, and/or the subject's condition, including the type and severity of the HIV infection, and can be determined by the skilled practitioner. The disclosed binding proteins can be formulated in a pharmaceutical composition suitable for administration to a subject by any intended route of administration, as discussed in more detail below.

### VI. Methods of Making Binding Proteins

While the disclosed binding proteins may be prepared using any known method of protein or antibody production, they also can be prepared using the methodologies disclosed herein. In particular, human neutralizing monoclonal antibodies or binding protein can be produced according to the following processes, rather than “humanizing” mouse or rat antibodies/peptides. In general, this process allows for the development of an effective, strong, and robust library of biologics (e.g., binding proteins) that have pharmaceutical applications with significant benefits to patients or animals in the global marketplace.

Using a parent hybridoma cell line, any one or more of four distinct and effective products can be produced: (1) a fully human neutralizing monoclonal antibody-directed against any pathogen (e.g., virus or bacteria)-through use in passive immunotherapy; (2) an effective humoral active vaccine that is safe and effective; (3) an oral mini-antibody peptide-based medication with an efficacy that is equivalent to the immunologic capacity of the monoclonal antibody produced by a parent hybridoma cell; and (4) an entry-fusion inhibitor that is immunologic in character and scope. The applications for these products are broad, effective and beneficial for therapeutic use. For example, monoclonal antibodies for therapeutic use may be made to treat viruses, including retroviruses or lentiviruses like HIV, among others.

In some embodiments, the disclosed method of producing a binding protein or antibody against any one of the disclosed antigens or epitopes (any one of SEQ ID NOs: 1-7) may comprise the steps of: (a) identifying an asymptomatic patient after natural infection by a target infectious agent (e.g., HIV or another lentivirus) as a donor for obtaining immune B-lymphocytes that produce high titers of plasma neutralizing antibodies directed against the target infectious agent; (b) collecting B-lymphocytes from the patient; (c) immortalizing the human B-lymphocytes to obtain immortalized cell lines; and (d) collecting antibodies produced by the immortalized cell lines. This process may optionally include the steps of (e) stabilizing and augmenting neutralizing antibody production by the immortalized cells lines; (f) screening supernatants from the immortalized cell lines for antibody production; and (g) testing the antibodies for binding against protein components of the infectious agent. The method may further comprise one or more of epitope mapping the antibodies that tested positive for binding to the infectious agent to screen for antibodies/binding proteins that specifically bind to any one of SEQ ID NOs: 1-7; purifying the antibodies by affinity chromatographic techniques; and in vitro testing of the antibodies to confirm neutralization reactivity against the target infectious agent at physiologic concentrations.

### VII. Pharmaceutical Compositions

Also provided herein are pharmaceutical compositions comprising a disclosed antigen (SEQ ID Nos. 1-7) (e.g., for a vaccine composition) or a disclosed binding protein (specifically binding to SEQ ID Nos. 1-7) (e.g., for a passive immunization or therapeutic composition) and a pharmaceutically acceptable carrier or diluent. A vaccine composition as disclosed herein may include one or a plurality of peptide antigens, each comprising or consisting of a different one of SEQ ID NOs: 1-7, including one, two, three, four, five, six, or seven of SEQ ID NOs: 1-7, and, optionally SEQ ID NO:8. A passive immunization composition as disclosed herein may include one or a plurality of binding proteins, each binding to a different one of SEQ ID NOs: 1-7, including binding proteins binding to one, two, three, four, five, six, or seven of SEQ ID NOs:1-7, and, optionally, a binding protein binding to SEQ ID NO:8. Additionally or alternatively, a passive immunization composition may comprise a plurality of binding proteins (i.e., antibodies) that each bind to the same one of SEQ ID NOs: 1-7. As noted above, in any embodiments, the binding proteins may be monoclonal antibodies.

The disclosed pharmaceutical compositions may be formulated for any suitable route of administration, including intravenous, subcutaneous, intraperitoneal, intramuscular, or oral administration. In typical embodiments, the binding proteins are formulated for intravenous, subcutaneous, intraperitoneal, or intramuscular administration, such as in a solution, suspension, emulsion, liposome formulation, etc. More specifically, the disclosed binding proteins can be formulated for intravenous, subcutaneous, or intramuscular administration. The pharmaceutical composition can be formulated to be an immediate-release composition, sustained-release composition, delayed-release composition, etc., using techniques and excipients that are known in the art.

Pharmaceutically acceptable carriers for various dosage forms and routes of administration are known in the art. For example, solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

Pharmaceutical compositions of the disclosed antigens or binding proteins can be prepared as formulations according to standard methods (see, for example, Remington's Pharmaceutical Science, Mark Publishing Company, Easton, USA). The pharmaceutical compositions generally comprise a carrier and/or additive in addition to the antigen or binding protein (e.g., antibody). For example, the pharmaceutical composition may comprise one or more surfactants (for example, PEG and Tween), excipients, antioxidants (for example, ascorbic acid), preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, corrigents, light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. The pharmaceutical composition may comprise one or more other low-molecular-weight polypeptides or proteins, such as serum albumin, gelatin, immunoglobulin, or amino acids such as glycine, glutamine, asparagine, arginine, and lysine.

When the antigens or binding proteins are prepared as an aqueous solution for injection, the antigen or binding protein may be formulated in an isotonic solution containing, for example, physiological saline, dextrose, or other excipients or tonifiers. The tonifier may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

The disclosed antigens and binding proteins may be formulated for administration by injection or infusion, such as an intravenous injection or infusion, an intramuscular injection, or a subcutaneous injection. Alternatively, the disclosed antigens or binding proteins may be formulated for oral administration.

Any of the pharmaceutical compositions disclosed herein can be used for inducing an immune response to, or treating and/or preventing a HIV infection, such as AIDS. Optimal doses and routes of administration may vary, such as based on the route of administration and dosage form, the age and weight of the subject, and/or the subject's condition, including the severity of the HIV infection, and can be determined by the skilled practitioner.

### VIII. Treatment and Prevention of HIV Infection

HIV is highly transmissible and causes serious infections that can be debilitating and lead to death. The present disclosure provides methods of treating or preventing HIV and uses of binding proteins (e.g., antibodies) and vaccines based on the highly conserved antigens and epitopes disclosed herein.

The present disclosure provides methods of inducing an immune response against HIV by administering an antigen as disclosed herein (SEQ ID NOs: 1-7) in the form of a vaccine as discussed above. The present disclosure also provides uses of the disclosed antigens and pharmaceutical compositions (e.g., vaccines) for inducing an immune response to HIV. The immune response may be effective to reduce the risk of infection by HIV and the development of AIDS. The immune response may be effective to partially or fully protect against infection, such as by preventing infection or reducing the viral load if the subject does get infected.

The present disclosure also provides methods of treatment and prevention of HIV infections (e.g., AIDS) by administering a binding protein that specifically binds to at least one of the epitopes disclosed herein (SEQ ID NOs: 1-7), optionally with a binding protein that specifically binds SEQ ID NO:8. The present disclosure also provides uses of the disclosed binding proteins and pharmaceutical compositions for treating or preventing HIV infections (e.g., AIDS). As noted above, in any embodiments, the binding proteins may be monoclonal antibodies.

The disclosed methods comprise administering to a subject an effective amount of one or more of the antigens or binding proteins or pharmaceutical compositions disclosed herein. Administration may be performed via intravenous, intra-arterial, intramuscular, subcutaneous, or intradermal injection. In some embodiments, the subject may be at risk of exposure to a HIV. In some embodiments, the administration of the antigen prevents the subject from developing a HIV infection and/or AIDS. In some embodiments, the administration of the antigen reduces the risk the subject will develop a severe HIV infection (e.g., AIDS), such as reducing the risk of infection requiring hospitalization, reducing the risk of transmission, or reducing the need for additional treatment or prophylaxis. In some embodiments, the subject may have previously been exposed to HIV. In some embodiments, particularly embodiments using binding proteins, the subject may have an active infection (e.g., AIDS) which may be treated as a result of the administration. In some embodiments, the administration of the binding protein prevents the subject from developing a HIV infection and/or AIDS. In some embodiments, the effective amount of a binding protein is sufficient to reduce circulating viral load and/or to reduce, ameliorate, or eliminate one or more symptoms or effects of a HIV infection. In some embodiments, the effective amount of a binding protein is effective to prevent binding of a HIV gp41 protein to a host cell. The specific amount of antigen or binding protein administered may depend on one or more of the age and/or weight of the subject and/or the stage or severity of the disease and/or the dosage form and route of administration, and can be determined by the skilled practitioner.

The disclosed methods and use for treating, preventing, and/or reducing the risk of HIV infection or AIDS described herein comprise administering to a mammalian subject in need thereof a HIV-binding peptide as disclosed herein, or a pharmaceutical composition comprising the same. In some embodiments, the methods comprise administering a HIV-binding peptide to a subject that is at risk of becoming infected with HIV, has been infected with HIV (e.g., the patient has a Stage 1 or Stage 2 HIV infection), or has developed AIDS. In some embodiments, the methods may comprise administering both a HIV-binding peptide and another compound that is useful for treating HIV/AIDS, such as one or more anti-retroviral drugs such as TDF (tenofovir), 3TC (lamivudine), FTC (emtricitabine), or EFV (efavirenz). In such embodiments, the HIV-binding peptide and the other compound(s) can be administered sequentially or simultaneously, from the same or different compositions. Thus, treatment may include administering antiretroviral drug(s) and/or other supportive treatments to address the symptoms and/or effects of HIV infection or AIDS.

For the purposes of the disclosed methods and uses, treatment and/or prevention of all strains and variants of HIV are specifically contemplated.

Dosage regimens can be adjusted to provide the optimum desired response. For example, in some embodiments, a single bolus of an antigen or binding protein may be administered, while in some embodiments, several doses may be administered over time, or the dose may be proportionally reduced or increased as indicated by the situation. In some embodiments, a subject may be administered more than one distinct antigen or binding protein, such as two or three or more distinct antigens or antibodies that each bind to different epitopes disclosed herein.

### IX. Detection of HIV Infection

As noted above the peptide antigens and binding proteins thereto described herein also are useful for detecting HIV infection or AIDS.

In general, detection tests can be classified into two categories: diagnostic tests and surveillance tests. Diagnostic tests detect a component of the virus in a sample, typically taken from the nasal cavity, throat, saliva, blood, plasma, or serum. The test format can be a molecular test that detects viral RNA or an antigen test that detects viral protein. Molecular tests also are called Nucleic Acid Amplification Tests (NAATs), and involve amplifying nucleic acids present in the sample until they are detectable. The polymerase chain reaction (PCR) is viewed as the “gold standard” for diagnostic tests, but can show false-negative results at early stages of infection. RT-PCR is expensive, requires expert handling, and takes about four hours to complete the assay.

The mutagenic rate of HIV has resulted in variants having mutations in their genomic and protein sequences, and this poses another problem in the development of effective methods for detecting infection. Mutations can impact test performance if the mutation impairs or prevents the test reagent from being able to detect the virus. The impact of mutations on a test's performance may be influenced by several factors, including the sequence of the variant (including the number, identity and location of mutations), the design of the test, and the prevalence of the variant in the population. For example, tests with single targets are more likely to fail to detect new variants. On the other hand, tests with multiple targets (e.g., a PCR test designed to detect more than one section of the HIV genome or an antigen test designed to detect more than one region of the spike protein) are more likely to be able to detect new variants.

The peptide antigens and binding proteins described herein offer significant advantages in this context due to the highly conserved nature of the corresponding epitopes. As discussed above, the epitopes of SEQ ID NOs: 1-7 have been confirmed to be conserved across 87,336 HIV isolates, and these sequences are linear and accessible to antibodies. This indicates that detection tests targeting these epitopes will be able to detect infection by all currently known variants, as well as future variants. Disclosed tests can involve detecting any one or more or all of SEQ ID NOs: 1-7 using a binding protein or, alternatively, can involve detecting antibodies that bind to any one or more or all of SEQ ID NOs: 1-7, as described herein. In any embodiments, SEQ ID NO:8 or antibodies that bind to SEQ ID NO:8 also may be detected.

Thus, provided herein are an “antigen test” and kit for detecting HIV infection, comprising detecting any one or more or all of SEQ ID NOs: 1-7 in a sample obtained from a subject, including detecting any one, two, three, four, five, six, or seven of SEQ ID NOs: 1-7 in a sample. For the purposes of such antigen tests, an ELISA (i.e., enzyme-linked immunoassay) is a particularly useful format, but other known methods in the art may be used, such as Western blotting, dot blotting, immunohistochemistry, immunofluorescence, immunoprecipitation, immunoelectrophoresis, or mass-spectrometry, and any other assay format that can detect the presence of any one or more or all of SEQ ID NOs: 1-7 in a sample. In any embodiments, SEQ ID NO:8 also may be detected.

For example, an ELISA-based method of detecting HIV infection may comprise contacting a sample obtained from a subject with one or more probe binding proteins or antibodies that each specifically bind any one or more or all of SEQ ID NOs: 1-7, and detecting binding between the probe binding proteins/antibodies and any HIV antigen present in the sample that comprises or consists of these sequences.

As noted above, in some embodiments, the methods may also comprise contacting the sample with one or more antibodies that binds to a peptide antigen comprising or consisting of KLIC (SEQ ID NO: 8), with corresponds to amino acids 601-604 of gp41. KLIC (SEQ ID NO:8) was observed to be conserved in 73.083% of the 87,336 HIV isolates that were analyzed.

In some embodiments, a method may comprise using a single monoclonal antibody that is specific for any one of SEQ ID NOs: 1-7 or a panel of binding proteins/antibodies that each specifically binds one of SEQ ID NOs: 1-7 and, optionally, SEQ ID NO: 8, typically where each specifically binds a different one of SEQ ID NOs: 1-7 (or SEQ ID NO: 8), such as a panel of 2-8 (e.g., 2, 3, 4, 5, 6, 7, or 8) binding proteins/antibodies, where each specifically binds a different one of SEQ ID NOs: 1-7 and, optionally, SEQ ID NO: 8. In such embodiments, the probe antibodies or binding proteins that bind to the HIV antigens may be bound to a solid substrate (e.g., a plate, well, slide, bead, strip, etc.) and a biological sample obtained from a subject (such as an individual suspected of having or having been exposed to HIV) may be applied to the substrate. If a target antigen (i.e., antigens comprising or consisting of SEQ ID NOs: 1-7 and, optionally, SEQ ID NO: 8) is present in the sample and a probe antibody/binding protein that is specific for the target antigen is bound to the substrate, then the target antigen will be bound. The biological sample can then be removed and the substrate may be washed to remove any unbound protein or debris. Next, a detection antibody that also binds to the target antigen may be contacted to the substrate. The detection antibody typically is detectably labeled, such that it can be detected as evidence of the presence of the target antigen in the sample. Detectable labels that can be used for this purpose are known in the art and can include, but are not limited to, a fluorophore (e.g., FTIC, rhodamine, GFP, lanthanide, etc.), a chromogen, a chemiluminescent agent, an enzymatic label (e.g., luciferase, horseradish peroxidase, alkaline phosphatase), an acridinium moiety, a radiolabel, a colorometric label, a magnetic agent, or a metal (e.g., a gold particle).

Also provided herein is an antibody test for detecting HIV infection, comprising detecting antibodies to any one or more or all of SEQ ID NOs: 1-7 in a sample obtained from a subject, including antibodies to any one, two, three, four, five, six, or seven of SEQ ID NOs: 1-7 in a sample. Antibodies to each of SEQ ID NOs: 1-7 can be detected using a peptide antigen as described herein as a probe. In some embodiments, the method further comprises detecting an antibody that binds to a peptide antigen comprising or consisting of KLIC (SEQ ID NO: 8). Thus, a method of detecting SARS-CoV-2 infection may comprise contacting a sample obtained from a subject with one or more peptide antigens, each comprising or consisting of one of SEQ ID NOs: 1-7, and, optionally, SEQ ID NO: 8. In some embodiments, such a method comprises using a panel of peptide antigens, each comprising or consisting of one of SEQ ID NOs: 1-7, or, optionally, SEQ ID NO: 8, typically where each comprises or consists of a different one of SEQ ID NOs: 1-7 (or 8), such as a panel of 2-8 (e.g., 2, 3, 4, 5, 6, 7, or 8) peptide antigens, typically where each comprises or consists of a different one of SEQ ID NOs: 1-7 and, optionally, SEQ ID NO: 8. For the purposes of such embodiments, the probe antigen(s) may be bound to a substrate (e.g., a plate, well, slide, bead, strip, etc.) either directly or indirectly (e.g., via a polymer, such as polyethylene glycol (PEG), a peptide linker, or a protein, such as an antibody). The substrate may be contacted with a biological sample obtained from a subject (such as an individual suspected of having or having been exposed to HIV). If there are antibodies present in the sample that bind to any one of the probe antigens, then the presence of such antibodies can be detected, such as by removing the sample and washing the substrate to remove any unbound protein or debris, and then contacting the substrate with a detection antibody that binds to antibodies from the sample (e.g., human IgE or IgD). The detection antibody typically is detectably labeled, as discussed above.

The present disclosure also provides methods for detection of HIV by detecting the presence of nucleic acid sequence(s) that encode any one or more or all of SEQ ID NOs: 1-7. In some embodiments, the methods my further comprise detecting the presence of a nucleic acid sequence that encodes SEQ ID NO: 8. Such methods include, but are not limited to, RT-qPCR, RT-PCR, RNA-seq, Northern blotting, Serial Analysis of Gene Expression (SAGE), or DNA or RNA microarrays. The starting material for detection of polynucleotides encoding the disclosed biomarkers may be genomic DNA, cDNA, RNA or mRNA. In such embodiments, nucleic acid primers and, optionally, nucleic acid probes, may be designed to specifically amplify and detect the nucleic acid sequences that encode any one or more or all of SEQ ID NOs: 1-7 and, optionally, SEQ ID NO: 8. Primers and probes may comprise a detectable label or a plurality of detectable labels. The detectable label associated with the primer or probe can generate a detectable signal directly. Additionally, the detectable label associated with the primer or probe can be detected indirectly using a reagent, wherein the reagent includes a detectable label, and binds to the label associated with the probe.

Detectably labeled nucleic acid primers and probes can be used to monitor the amplification of a target nucleic acid sequence (e.g., nucleic acid sequences that encode any one or more or all of SEQ ID NOs: 1-7 and, optionally, SEQ ID NO: 8). In some embodiments, detectably labeled primers or probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5@; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Nucleic acid primers or probes may be designed to selectively hybridize to any portion of a nucleic acid sequence encoding any one or more or all of SEQ ID NOs: 1-7 and, optionally, SEQ ID NO: 8. Methods for preparing nucleic acid primers or probes are well known in the art.

Accordingly, the present disclosure provides in vitro methods of analyzing a biological sample obtained from a subject, comprising contacting the sample with a binding protein that specifically binds to a HIV peptide antigen selected from SEQ ID NOs: 1-7 and, optionally, SEQ ID NO:8, and detecting binding between the binding protein and any HIV antigen present in the sample. The methods may comprise contacting the sample with a panel of from 2 to 8 (e.g., 2, 3, 4, 5, 6, 7, or 8) binding proteins that each specifically binds to a different HIV peptide antigen selected from SEQ ID NOs: 1-7 and, optionally, SEQ ID NO: 8, and detecting binding between the binding proteins and any HIV antigen present in the sample.

The present disclosure also provides in vitro methods of analyzing a biological sample obtained from a subject, comprising contacting the sample with a HIV peptide antigen comprising or consisting of an amino acid sequence selected from SEQ ID NOs: 1-7 and, optionally, SEQ ID NO: 8, and detecting binding between the peptide antigen and any anti-HIV antibodies present in the sample. The methods may comprise contacting the sample with a panel of from 2 to 8 (e.g., 2, 3, 4, 5, 6, 7, or 8) peptide antigens each comprising or consisting of a different an amino acid sequence selected from SEQ ID NOs: 1-7 and, optionally, SEQ ID NO: 8, and detecting binding between the peptide antigen and any anti-HIV antibodies present in the sample.

The present disclosure also provides in vitro methods of analyzing a biological sample obtained from a subject, comprising extracting nucleic acids from the biological sample, contacting the extracted nucleic acids with a pair of primers that specifically amplify a nucleic acid sequence encoding any one of SEQ ID NOs: 1-7 and, optionally, SEQ ID NO: 8, and detecting the presence of the amplified nucleic acid sequence if present in the sample. The methods may comprise contacting the sample with a panel of from 2 to 8 (e.g., 2, 3, 4, 5, 6, 7, or 8) primer pairs each specific for a different nucleic acid sequence that encodes an amino acid sequence selected from SEQ ID NOs: 1-7 and, optionally, SEQ ID NO: 8, and detecting amplification of each nucleic acid sequence if present in the sample.

As discussed above, the epitopes of SEQ ID NOs: 1-7 to be targeted for detection can be selected depending on the aim of the analysis.

In some embodiments of the disclosed methods, the sample may be selected from saliva, nasal fluid, nasal cells, throat cells, blood, plasma, serum, urine, and feces. However, the sample is not necessarily limited to these sample types. In some instances, a blood sample, a plasma sample, a serum sample, or a tissue sample may be appropriate. In some instances, the sample may comprise urine or feces, which may be useful for epidemiological studies and public health tracking that relies on wastewater. Thus, in some embodiments the sample comprises biological samples obtained from a plurality of subjects.

In some embodiments of the disclosed methods, the subject is suspected of having a HIV infection, has been exposed to HIV, or is suspected of having been exposed to HIV. In some embodiments of the disclosed methods, the method may further comprise determining that the subject is infected with HIV when binding or amplification is detected.

As noted above, in some embodiments, the sample comprises biological samples obtained from a plurality of subjects. Some such embodiments further comprise determining a level of infection in the plurality of subjects.

The present disclosure additionally provides kits for implementing any of the foregoing methods of detection.

For example, the present disclosure provides kits comprising at least one binding protein that specifically binds to a peptide comprising or consisting of any one of SEQ ID NOs: 1-7, a solid substrate to which the at least one binding protein is attached, and a second detectably labeled antibody that specifically binds to the peptide to which the at least one binding protein specifically binds. In some embodiments, the kit may further comprise a binding protein that specifically binds to a peptide comprising or consisting of SEQ ID NO: 8. Similarly, the present disclosure provides kits comprising at least one peptide comprising or consisting of any one of SEQ ID NOs: 1-7, a solid substrate to which the at least one peptide is attached, and a detectably labeled antibody that specifically binds to IgE or IgD, wherein the IgE or IgD are optionally human. In some embodiments, the kit may further comprise a peptide comprising or consisting of SEQ ID NO: 8. Similarly, the solid substrate can be selected from a bead, a plate, a well, a dish, a slide, or a strip.

The present disclosure also provides kits comprising at least one primer pair capable of specifically amplifying a nucleic acid sequence that encodes a peptide selected from any one of SEQ ID NOs: 1-7, wherein: (a) at least one primer of the primer pair is detectably labeled; or (b) the kit further comprises a detectably labeled probe that hybridizes to the nucleic acid sequence amplified by the primer pair. In some embodiments, the kit may comprise 1, 2, 3, 4, 5, 6, or 7 primer pairs, each primer pair being capable of specifically amplifying a different peptide selected from SEQ ID NOs: 1-7, wherein: (a) at least one primer of each primer pair is detectably labeled; or (b) the kit further comprises a detectably labeled probe that hybridizes to each nucleic acid sequence amplified by primer pairs included therein. In some embodiments, the kits may further comprise a primer pair that hybridizes to or is capable of amplifying a nucleic acid encoding SEQ ID NO: 8. In some embodiments, the kits may also comprise a detectably labeled probe that hybridizes to SEQ ID NO: 8.

The following examples are given to illustrate the present disclosure. It should be understood that the invention is not to be limited to the specific conditions or details described in these examples.

### EXAMPLES

### Example 1—Identification of Highly Conserved Antigens and Epitopes

This example describes the analytical methods used to identify the disclosed putative epitopes and antigens. The identification process used computational prediction and machine learning (ML) approaches to identify optimal targets and assess antigenicity.

Data Collection

A protein antigen dataset was created to train an artificial intelligence model to predict and map other linear, sequential, conserved and neutralizable B-cell epitopes within the HIV gp41 protein. HIV gp41 protein sequences were collected and multiple sequence alignments were performed to identify conserved sequences. Additional epitope databases (e.g., IEDB and AntiJen, BciPep, Epitome, SDAP, FLAVIdB, and Influenza Sequence and Epitope Database) were used to obtain independent and relevant data points to ensure unbiased training.

Curating Dataset

All identical epitopes (sequence and structure based) and non-immunogenic peptides were removed, so as to achieve unique experimentally proved epitopes. Generally, majority of B-cells epitope have length less than or equal to 20 amino acid, hence in this analysis all the epitopes having length more than 20 residues were removed. For training of machine learning technique, it was necessary to have fixed length patters whereas B-cell epitopes have varying length, hence if the epitope length were less than 20 amino acids, then the length was increased by introducing equal number of residues at both terminals derived from its original antigenic sequence. To generate a negative dataset, non-epitopes were created using random peptides of length 20 residues from the proteins in Swiss-Prot. All the random peptides that are identical to B-cells epitopes were excluded. Next, the unique epitope data was scored based on biophysical and biochemical metrics such as computed volume, polarity, hydrophobicity, linearity, activity and immunogenicity. These metrics served as dimensions across which regression, classification methods were performed. After a cleaned and labelled dataset was generated a portion of the dataset was used for training purposes. In order to achieve five-fold cross-validation approach. Multilayer perceptron method was implemented. The dataset was randomly divided into five subsets each containing an equal number of epitopes data. The three of the subsets were used for training purpose and from remaining two subsets each subset was used for validation and testing. This process was repeated five times so that each set was used once for testing. The final prediction results gave the average of five testing sets.

ANN Model Training

To predict the probability that a given antigen residue is part of an epitope, artificial neural network (Jordon Network) was implemented using Keras and TensorFlow. The networks were trained using back-propagation algorithm and with various window lengths from 10 to 20 residues. The target output consists of a single binary number with one or zero (B-cell epitopes or non-epitopes). At the beginning of each simulation, the weights were initialized with random values and the training was carried out by using error back-propagation, with a sum of square error function. In each cycle of the training, the magnitude of the error sum in the test and training set were monitored and the ultimate number of cycles were determined when the network converges. Also a cut off value for each network was set up, which was used to compare the output produced by the network. Thus, when the output value was greater than the threshold value, then that peptide was predicted as B-cell epitope, otherwise as a non-epitope. Additionally, each amino acid composition, along with other parameters such as computed volume, polarity, hydrophobicity, linearity, neutralizing activity and immunogenicity were used for prediction purpose. We accurately predict the likelihood of each residue being an epitope candidate. To ensure the capability of the model in determining the correct epitope for a given antibody, these models with two hidden layers were implemented. Parameters of prediction include amino acid composition, exposed donors/receptors, hydrophobicity, aromatic/positive/negative residues, size, antigen patch density and structural conjoint triads to represent the specified protein sequences by considering not only the composition of amino acids but also the neighbor relationships in that sequence. Once the system was trained on the curated database, the blind dataset evaluation was performed using 1) clinically validated immunogenic proteins, 2) IgE epitopes of allergenic proteins (SDAP), and none of these datasets were used in the training or testing.

Statistical Evaluation of Linear Epitope Dataset

FASTA sequences of the HIV peptides were inputted into the system and epitope predictions with percentage of conservedness, neutralizing activity and immunogenicity were established. Each model was tuned by comparing prediction accuracy from the test set to predict the accuracy on the validation set. Models that perform the best were scored on the test set and evaluated using metrics such as precision, recall, true positive rate, false positive rate, and ROC-area under the curve using sci-kitlearn, Keras, and TensorFlow. Once potential candidates were identified, the performance for each antigen in terms of the area under the receiver operation curve, the positive predictive rate and the true positive rate of the top predictions was evaluated for immune recognition.

Thus, SEQ ID NOs: 1-7 were identified as highly conserved epitopes having the conservation across isolates reported in Table 1 and FIG. 1.

What is claimed is:

1. An isolated peptide antigen comprising or consisting of an amino acid sequence of any one of SEQ ID NOs: 1-7.

2. The isolated peptide antigen of claim 1, wherein the peptide is recombinant.

3. A vaccine composition comprising a peptide antigen of claim 1 or claim 2 and a pharmaceutically acceptable carrier.

4. An isolated binding protein that binds to a conserved epitope of a HIV gp41 protein or gp120 protein, wherein the conserved epitope comprises the amino acid sequence of any one of SEQ ID NOs: 1-7.

5. The isolated binding protein of claim 4, wherein the binding protein prevents binding of HIV to a host cell.

6. The isolated binding protein of claim 4 or 5, wherein the binding protein is an antibody or an antibody fragment.

7. A method of reducing the risk of a HIV infection in a subject, comprising administering to the subject an effective amount of an isolated peptide antigen according to claim 1 or claim 2 or a vaccine according to claim 3.

8. A method of treating, preventing, or reducing the risk of a HIV infection in a subject, comprising administering to the subject a therapeutically effective amount of an isolated binding protein according to any one of claims 4-6.

9. The method of claim 7 or 8, wherein the isolated peptide antigen or isolated binding protein is administered by subcutaneous or intramuscular injection.

10. An isolated peptide antigen according to any one of claims 1-2 or vaccine according to claim 3, for inducing an immune response to HIV.

11. An isolated binding protein according to any one of claims 4-6 for treating, preventing, or reducing the risk of a HIV infection in a subject in need thereof.

12. Use of an isolated peptide antigen according to any one of claims 1-2 in the preparation of a vaccine for inducing an immune response to HIV.

13. Use of an isolated binding protein according to any one of claims 4-6 in the preparation of a medicament for treating, preventing, or reducing the risk of a HIV infection in a subject in need thereof.

14. A method of preparing an antibody that binds to a peptide antigen of claim 1 or 2, comprising:
(a) identifying an asymptomatic patient that has been infected with HIV as a donor for obtaining immune B-lymphocytes that produce high titers of HIV-neutralizing antibodies;
(b) collecting the B-lymphocytes from the patient;
(c) immortalizing the B-lymphocytes;
(d) collecting antibodies produced by the immortalized B-lymphocytes; and
(e) screening the antibodies for binding to the peptide antigen of claim 1 or 2.

15. The method of claim 14, further comprising testing the antibodies for binding to HIV.

16. The method of claim 15, further comprising epitope mapping the antibodies that tested positive for binding to HIV.

17. The method of any one of claims 14-16, wherein immortalizing the B-lymphocytes comprises fusing a B-lymphocyte with a heteromyeloma cell in order to produce a heterohybridoma cell.

18. An in vitro method of analyzing a biological sample obtained from a subject, comprising contacting the sample with a binding protein that specifically binds to a HIV peptide antigen selected from SEQ ID NOs: 1-7, and detecting binding between the binding protein and a HIV antigen present in the sample.

19. The method of claim 18, comprising contacting the sample with a panel of from 2 to 7 binding proteins that each specifically binds to a different HIV peptide antigen selected from SEQ ID NOs: 1-7, and detecting binding between the binding proteins and a HIV antigen present in the sample.

20. The method of claim 18 or 19 further comprising contacting the sample with a binding protein that specifically binds to SEQ ID NO: 8.

21. An in vitro method of analyzing a biological sample obtained from a subject, comprising contacting the sample with a HIV peptide antigen comprising or consisting of an amino acid sequence selected from SEQ ID NOs: 1-7, and detecting binding between the peptide antigen and any anti-HIV antibodies present in the sample.

22. The method of claim 21, comprising contacting the sample with a panel of from 2 to 7 peptide antigens each comprising or consisting of a different an amino acid sequence selected from SEQ ID NOs: 1-7, and detecting binding between the peptide antigen and any anti-HIV antibodies present in the sample.

23. The method of claim 21 or 22 further comprising contacting the sample with a peptide antigen comprising or consisting of SEQ ID NO: 8.

24. An in vitro method of analyzing a biological sample obtained from a subject, comprising extracting nucleic acids from the biological sample, contacting the extracted nucleic acids with a pair of primers that specifically amplify a nucleic acid sequence encoding a peptide of any one of SEQ ID NOs: 1-7, and detecting the presence of the amplified nucleic acid sequence if present in the sample; optionally, wherein the peptide comprises or consists of any one of SEQ ID NOs: 1-7.

25. The method of claim 24, comprising contacting the sample with a panel of from 1 to 7 primer pairs that each specifically amplify a nucleic acid sequence that encodes a different amino acid sequence selected from SEQ ID NOs: 1-7, and detecting amplification of each nucleic acid sequence if present in the sample.

26. The method of claim 24 or 25, further comprising contacting the sample with a primer pair that specifically amplifies a nucleic acid sequence encoding a peptide comprising or consisting of SEQ ID NO: 8.

27. The method of any one of claims 18-26, wherein the sample is selected from saliva, nasal fluid, nasal cells, throat cells, blood, plasma, serum, urine, and feces.

28. The method of any one of claims 18-27, where the subject is suspected of having a HIV infection, has been exposed to HIV, or is suspected of having been exposed to HIV.

29. The method of any one of claims 18-28, further comprising determining that the subject is infected with HIV when binding or amplification is detected.

30. A kit comprising at least one binding protein that specifically binds to a peptide comprising or consisting of any one of SEQ ID NOs: 1-7, a solid substrate to which the at least one binding protein is attached, and a detectably labeled antibody that specifically binds to the peptide to which the at least one binding protein specifically binds; optionally, wherein the kit further comprises a binding protein that specifically binds to a peptide comprising or consisting of SEQ ID NO: 8 attached to the solid support.

31. A kit comprising at least one peptide comprising or consisting of any one of SEQ ID NOs: 1-7, a solid substrate to which the at least one peptide is attached, and a detectably labeled antibody that specifically binds to IgE or IgD, wherein the IgE or IgD are optionally human;
optionally, wherein the kit further comprises a peptide comprising or consisting of SEQ ID NO:8 attached to the solid support.

32. A kit comprising at least one primer pair capable of specifically amplifying a nucleic acid sequence that encodes a peptide selected from any one of SEQ ID NOs: 1-7, wherein:
(a) at least one primer of the primer pair is detectably labeled; or
(b) the kit further comprises a detectably labeled probe that hybridizes to the nucleic acid sequence amplified by the primer pair;
optionally, wherein the kit further comprises a primer pair capable of specifically amplifying a nucleic acid sequence that encodes a peptide comprising or consisting of SEQ ID NO: 8.


SEQUENCE LISTING (8 sequences)

SEQ ID NO: 1 - protein, 5 aa, Human immunodeficiency virus 1
YGVPV

SEQ ID NO: 2 - protein, 5 aa, Human immunodeficiency virus 1
WVTVY

SEQ ID NO: 3 - protein, 10 aa, Human immunodeficiency virus 1
WVTVYYGVPV

SEQ ID NO: 4 - protein, 4 aa, Human immunodeficiency virus 1
RIRQ

SEQ ID NO: 5 - protein, 4 aa, Human immunodeficiency virus 1
LLGI

SEQ ID NO: 6 - protein, 4 aa, Human immunodeficiency virus 1
LLGR

SEQ ID NO: 7 - protein, 4 aa, Human immunodeficiency virus 1
QHLL

SEQ ID NO: 8 - protein, 4 aa, Human immunodeficiency virus 1
KLIC